(19) United States
(12) Patent Application Publication
(10) Pub. No.: US 2025/0201428 A1
(43) Pub. Date: Jun. 19, 2025

(54) SYSTEM AND A METHOD FOR REMOTE MEDICAL CONSULTATION

(71) Applicant: Cognota Healthcare Private Limited, Thane (IN)

(72) Inventor: Rajendra Patil, Pune (IN)

(21) Appl. No.: 18/985,189

(22) Filed: Dec. 18, 2024

(30) Foreign Application Priority Data
Dec. 18, 2023 (IN) 202321086625

(51) Int. Cl. G16H 80/00; G16H 10/20; G16H 10/60; G16H 20/60; G16H 40/20

(52) U.S. Cl. CPC G16H 80/00; G16H 10/20; G16H 10/60; G16H 20/60; G16H 40/20

(57) ABSTRACT

A system 100 for remote medical consultation is disclosed. The system includes a processing subsystem 102 which includes a communication module 108 configured to remotely communicate a patient 110 with a plurality of medical assistants, a medical assistant recommendation module 114 recommends a medical assistant to the patient by generating a list of medical assistants consulted by the patient and a list of the medical assistants without consultation by the patient, an examination module 116 allows the medical assistant to remotely examine a plurality of health parameters of the patient, a diet recommendation module 118 recommends a customized diet plan to the patient, a template generation module 120 generates a template by accessing a plurality of open platforms to allow the patient to fill a diagnostic data in a plurality of data fields, recommend the template to the patient based on an input from the medical assistant.

### EARLIEST PRIORITY DATE

This application claims priority from a complete patent application filed in India having patent application No. 202321086625, filed on 18th day of December 2023, and titled “A SYSTEM AND A METHOD FOR REMOTE MEDICAL CONSULTATION”.

### FIELD OF INVENTION

Embodiments of the present disclosure relate to medical services and more particularly to a system and a method for remote medical consultation.

### BACKGROUND

Patients often visit doctors or medical assistants to seek advice for their health problems. Also, doctors need the patients to be physically present to assess the patient's preferences for the desired diagnosis, learn their background information and medical history, discuss options and anticipated costs, collect signed consent forms releasing the physician of liability. At times, it is not possible for every patient to visit the doctor physically. Also, many specialized doctors live in different cities than patients hence it is not possible for bedridden or old age people to go to visit the doctor.

Currently, few existing systems facilitate patient-doctor communication by avoiding frequent visits to the doctor's place. But the current systems do not provide options for the medical assistants by displaying a list of medical assistants to the patient. Also, current systems do not provide medical assistance in multiple areas such as diet, medicine, and the like.

Hence, there is a need for a system and a method for remote medical consultation that addresses the aforementioned issues.

### Objective of the Invention

An objective of the present invention is to provide a remote access to a medical assistant or a doctor and convenience to a patient thus saving time and efforts of the patient for a physical visit.

Another objective of the present invention provides online communication tools and information-sharing platforms, thereby enabling more efficient interactions between doctors or medical assistants and patients and hence reduces the number of visits required and allows for quicker access to medical advice and services.

Yet, an objective of the present invention is to enable the collection, storage, and retrieval of patient information in an electronic database.

Further, an objective of the present invention is to facilitate patients to access specialized medical expertise by enabling remote consultations with physicians who may be located in different geographic regions.

### BRIEF DESCRIPTION

In accordance with one embodiment of the disclosure, a system for remote medical consultation is provided. The system includes a processing subsystem hosted on a server and configured to execute on a network to control bidirectional communications among a plurality of modules. The processing subsystem includes a communication module, a medical assistant recommendation module, an examination module, a template generation module, and a diet recommendation module. The communication module is configured to remotely communicate with a patient with a plurality of medical assistants. The medical assistant recommendation module is operatively connected to the communication module and configured to recommend a medical assistant from the plurality of medical assistants to a patient by generating a list of medical assistants consulted by the patient and a list of the medical assistants without consultation by the patient. The recommendation is based on the historical data of medical assistant. The examination module is operatively coupled with the medical assistant recommendation module and configured to allow the medical assistant to examine the patient remotely. The medical assistant is selected by the patient as recommended by a medical assistant recommendation module. The template generation module is operatively connected with the examination module. The template generation module is configured to generate a template to allow the patient to fill in diagnostic data in a plurality of data fields and access a plurality of open platforms for generating the template. The template generation module is configured to recommend the template to the patient based on an input from the medical assistant in a predefined data field. The recommended template comprises of one or more customizable pre-filled data fields. The diet recommendation module is operatively connected with the doctor recommendation module, wherein the diet recommendation module is configured to recommend a customized diet plan based on a plurality of health parameters of the patient.

In accordance with another embodiment, a method for operating a system for a remote medical consultation is provided. The method includes communicating, by a communication module of the processing subsystem, remotely by a patient with a plurality of medical assistants. The method includes recommending, by a medical assistant recommendation module of a processing subsystem, a medical assistant from the plurality of medical assistants to a patient by generating a list of medical assistants consulted by the patient and a list of the medical assistants without consultation by the patient, wherein the recommendation is based on the historic data of medical assistant. Further, the method includes remotely examining, by an examination module of the processing subsystem, a plurality of health parameters the patient, wherein the medical assistant is selected by the patient as recommended by a medical assistant recommendation module. Furthermore, the method includes recommending, by a diet generation module of the processing subsystem, a customized diet plan based on the plurality of health parameters of the patient. Furthermore, the method includes generating, by a template generation module of the processing subsystem, generate a template to allow the patient to fill a diagnostic data in a plurality of data fields and accessing a plurality of open platforms for generating the template. Furthermore, the method includes recommending, by the template generation module of the processing subsystem, the template based on an input from the medical assistant in a predefined data field. The recommended template comprises of one or more customizable pre-filled data fields.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram representing a system for remote medical consultation in accordance with an embodiment of the present disclosure;

FIG. 2 is a block diagram representing another embodiment of the system for remote medical consultation of FIG. 1 in accordance with an embodiment of the present disclosure;

FIG. 3 is a block diagram of a computer or a server for the system for remote medical consultation in accordance with an embodiment of the present disclosure; and

FIG. 4 is a flow chart representing steps involved in a method for operation of the system for remote medical consultation in accordance with an embodiment of the present disclosure.

Further, those skilled in the art will appreciate that elements in the figures are illustrated for simplicity and may not have necessarily been drawn to scale. Furthermore, in terms of the construction of the system, one or more components of the system may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure.

The terms “comprises”, “comprising”, or any other variations thereof, are intended to cover a non-exclusive inclusion, such that a process or method that comprises a list of steps does not include only those steps but may include other steps not expressly listed or inherent to such a process or method. Similarly, one or more devices or sub-systems or elements or structures or components preceded by “comprises . . . a” does not, without more constraints, preclude the existence of other devices, sub-systems, elements, structures, components, additional devices, additional sub-systems, additional elements, additional structures, or additional components. Appearances of the phrase “in an embodiment”, “in another embodiment”, and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

In the following specification and the claims, reference will be made to a number of terms, which shall be defined to have the following meanings. The singular forms “a”, “an”, and “the” include plural references unless the context clearly dictates otherwise.

Embodiments of the present disclosure relate to a system for remote medical consultation is provided. The system includes a processing subsystem hosted on a server and configured to execute on a network to control bidirectional communications among a plurality of modules. The processing subsystem includes a communication module, a medical assistant recommendation module, an examination module, a template generation module, and a diet recommendation module. The communication module is configured to remotely communicate with a patient with a plurality of medical assistants. The medical assistant recommendation module is operatively connected to the communication module and configured to recommend a medical assistant from the plurality of medical assistants to a patient by generating a list of medical assistants consulted by the patient and a list of the medical assistants without consultation by the patient. The recommendation is based on the historical data of medical assistant. The examination module is operatively coupled with the medical assistant recommendation module and configured to allow the medical assistant to examine the patient remotely. The medical assistant is selected by the patient as recommended by a medical assistant recommendation module. The template generation module is operatively connected with the examination module. The template generation module is configured to generate a template to allow the patient to fill in diagnostic data in a plurality of data fields and access a plurality of open platforms for generating the template. The template generation module is configured to recommend the template to the patient based on an input from the medical assistant in a predefined data field. The recommended template comprises of one or more customizable pre-filled data fields. The diet recommendation module is operatively connected with the doctor recommendation module, wherein the diet recommendation module is configured to recommend a customized diet plan based on a plurality of health parameters of the patient.

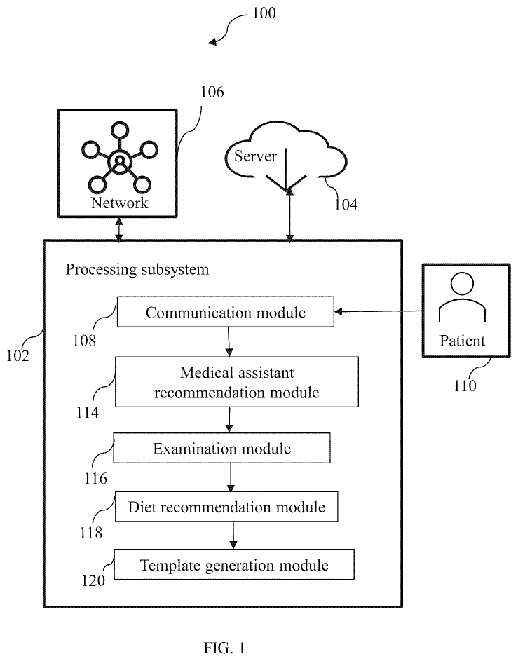

FIG. 1 is a block diagram representing a system 100 for remote medical consultation in accordance with an embodiment of the present disclosure. The system 100 includes a processing subsystem 102. The processing subsystem 102 is hosted on a server 104, wherein the processing subsystem 102 is configured to execute on a network 106 to enable communications among a plurality of modules. In one embodiment, the network 106 may include a wired network such as local area network (LAN). In another embodiment, the network 106 may include a wireless network such as Wi-Fi, Bluetooth, Zigbee, near field communication (NFC), infra-red communication (RFID) or the like. The plurality of modules includes a communication module 108, a medical assistant recommendation module 114, an examination module 116, a diet recommendation module 118, and a template generation module 120.

The communication module 108 is configured to remotely communicate with a patient 110 and with a plurality of medical assistants. In one embodiment, communication may be established through the Internet via a website. In one embodiment, the patient 110 is registered in the system 100 by logging in to the system 100.

The medical assistant recommendation module 114 is operatively connected to the communication module 108 and is configured to recommend a medical assistant from the plurality of medical assistants to the patient 110 by generating a list of medical assistants consulted by the patient 110 and a list of the medical assistants without consultation by the patient 110. The recommendation is based on the historical data of medical assistant. In one embodiment, the recommended medical assistants are registered on the system 100 via the website.

The examination module 116 is operatively coupled with the medical assistant recommendation module 114 and configured to allow the medical assistant to remotely examine a plurality of health parameters of the patient 110. The medical assistant is selected by the patient 110 as recommended by a medical assistant recommendation module 114. In one embodiment, the examination report may be stored in the database 128 as a history of the patient 110 and may be used for future treatment of the patient 110.

The diet recommendation module 118 is operatively connected with the examination module 116. The diet recommendation module 118 is configured to recommend a customized diet plan based on the plurality of health parameters of the patient 110. In one embodiment, the patient 110 can consult several medical assistants at the same time by using one login.

The template generation module 120 is operatively connected with the examination module 116. The template generation module 120 is configured to generate a template to allow the patient 110 to fill in diagnostic data in a plurality of data fields and access a plurality of open platforms for generating the template. The template generation module 120 is configured to recommend the template to the patient 110 based on an input from the medical assistant in a predefined data field, wherein the recommended template comprises of one or more customizable pre-filled data fields. In one embodiment, the template generation module 120 is configured to automatically generate the template in real-time and notify a user as a notification.

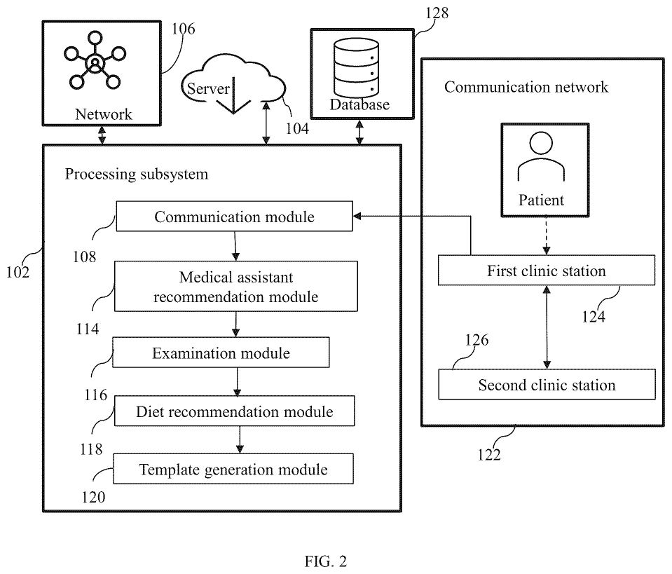

FIG. 2 is a block diagram representing another embodiment of the system for remote medical consultation in accordance with FIG. 1. The system 100 includes the processing subsystem 102 which further includes, a communication module 108, a medical assistant recommendation module 114, an examination module 116, a diet recommendation module 118, and a template generation module 120. In one embodiment, the system 100 includes a database 128 configured to store the data related to recommendations. The database 128 is accessible by the medical assistant.

In one embodiment, the system 100 includes a communication network 122. In one embodiment, the communication network 122 includes a first communication link between a first clinic station 124 and the patient 110, wherein the patient 110 requests for consultation to the first clinic station 124. The first communication link utilizes an interactive computer network. In another embodiment, the communication network 122 includes a second communication link between a second clinic station 126 and the first medical assistant for medical information exchange. Yet, in one embodiment, the communication network 122 is established through an internet website accessible by the patient 110. In one embodiment, the medical assistant at the first clinic station 124 evaluates the patient's condition by reviewing both sets of patients 110 information. Based on the assessment, the medical assistant may provide various consultation services, including diagnosis, prescription of medication, prescription of treatment, or referral to other medical professionals. The consultation service is communicated back to the patient station via the first communication link, allowing for remote provision of medical advice and care. In one embodiment the first clinic station 124 is operated by the medical assistant, a patient station located remotely from the first clinic station 124.

In one embodiment, the first clinic station 124 is located remotely from a patient station to communicate information relating to the patient 110. In one embodiment, the second clinic station 126 conducts an examination of the patient and collects a second set of information about the patient 110. In one embodiment, the second set of information may include interviews, physical examinations, and other necessary assessments of the patient 110. In one embodiment, the second clinic station 126 is operated by medical personnel for direct contact with the patient 110. The medical personnel are licensed medical assistants contracted with the assistance provider.

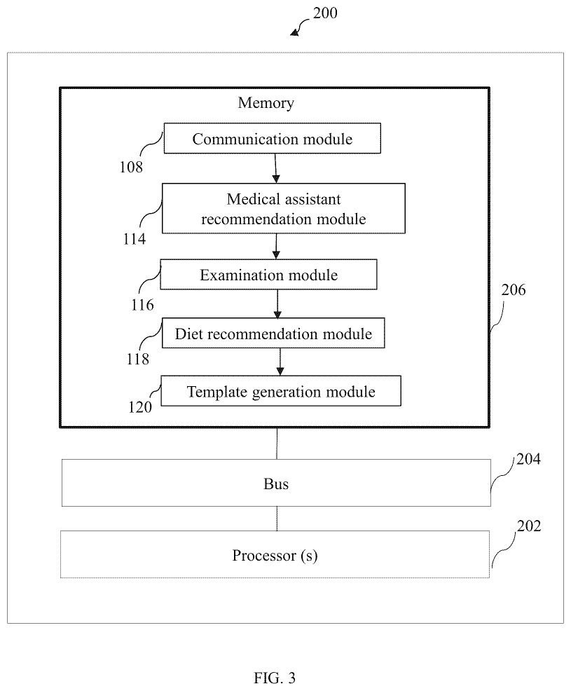

FIG. 3 is a block diagram 200 of a computer or a server for the system for remote medical consultation in accordance with an embodiment of the present disclosure. The server includes processor(s) 202, and memory 206 operatively coupled to the bus 204.

The processor(s) 202, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The bus 204 as used herein refers to internal memory channels or computer network that is used to connect computer components and transfer data between them. The bus 204 includes a serial bus or a parallel bus, wherein the serial bus transmits data in bit-serial format and the parallel bus transmits data across multiple wires. The bus 204 as used herein, may include but not limited to, a system bus, an internal bus, an external bus, an expansion bus, a frontside bus, a backside bus, and the like.

The memory 206 includes a plurality of subsystems and a plurality of modules stored in the form of executable program which instructs the processor 202 to perform the method steps illustrated in FIG. 1. The memory 206 is substantially similar to the system of FIG. 1. The memory 206 has following submodules: a communication module 108, a profile generation module 108, a medical assistant recommendation module 114, an examination module 116, a diet recommendation module 118, and a template generation module 120.

The communication module 108 is configured to remotely communicate with a patient 110 with a plurality of medical assistants.

The medical assistant recommendation module 114 is operatively connected to the communication module 108 and configured to recommend a medical assistant from the plurality of medical assistants to the patient 110 by generating a list of medical assistants consulted by the patient and a list of the medical assistants without consultation by the patient 110. The recommendation is based on the historical data of medical assistant.

Then examination module 116 operatively coupled with the medical assistant recommendation module 114 and configured to allow the medical assistant to remotely examine a plurality of health parameters of the patient. The medical assistant is selected by the patient 110 as recommended by a medical assistant recommendation module 114.

The diet recommendation module 118 operatively connected with the examination module 116. The diet recommendation module 118 is configured to recommend a customized diet plan based on the plurality of health parameters of the patient 110.

The template generation module 120 is operatively connected with the examination module 116. The template generation module 120 is configured to generate a template to allow the patient 110 to fill in diagnostic data in a plurality of data fields and access a plurality of open platforms for generating the template. The template generation module 120 is configured to recommend the template to the patient 110 based on an input from the medical assistant in a predefined data field, wherein the recommended template comprises of one or more customizable pre-filled data fields. In one embodiment, the template generation module 120 is configured to automatically generate the template in real-time and notify a user as a notification.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the processor(s) 202.

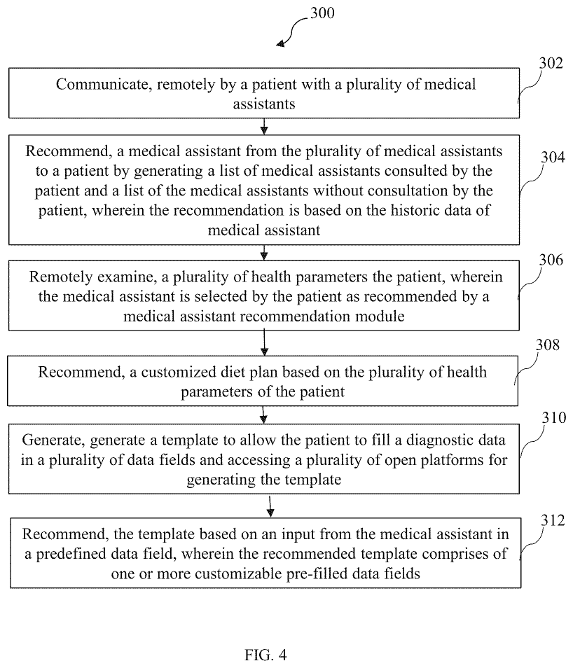

FIG. 4 is a flow chart representing steps involved in a method for operating a system 100 for remote medical consultation in accordance with an embodiment of the present disclosure. In one embodiment, the method starts with the patient station communicating remotely with the first clinic station and the physician for a first set of patient information. This communication occurs via the first communication link, often through an internet website accessible by the patient station. The physician then remotely interviews the patient or patient representative to gather additional information and establish a physician-patient relationship.

The method 300 includes communicating, by a communication module of the processing subsystem, remotely by a patient with a plurality of medical assistant's assistant in step 302. The method also includes registering, the patient in the system by logging in to the system.

The method 300 includes recommending, by a medical assistant recommendation module of a processing subsystem, a medical assistant from the plurality of medical assistants to a patient by generating a list of medical assistants consulted by the patient and a list of the medical assistants without consultation by the patient. The recommendation is based on the historical data of medical assistant in step 304.

Further, the method 300 includes remotely examining, by an examination module of the processing subsystem, a plurality of health parameters the patient, wherein the medical assistant is selected by the patient as recommended by a medical assistant recommendation module assistant in step 306. The method also includes storing, the examination report in the database as a history of the patient and may be used for future treatment of the patient.

Furthermore, the method 300 includes recommending, by a diet generation module of the processing subsystem, a customized diet plan based on the plurality of health parameters of the patient assistant in step 308.

Furthermore, the method 300 includes generating, by a template generation module of the processing subsystem, generate a template to allow the patient to fill a diagnostic data in a plurality of data fields and accessing a plurality of open platforms for generating the template assistant in step 310. The method includes generating, the template automatically in real-time and notify to a user as a notification.

Furthermore, the method 300 includes recommending, by the template generation module of the processing subsystem, the template based on an input from the medical assistant in a predefined data field assistant in step 312. The recommended template comprises of one or more customizable pre-filled data fields.

The method 300 also includes providing a communication network for remote communication. The method 300 also includes linking, by a first communication link, a first clinic station and the patient, wherein the patient requests for consultation to the first clinic station. The first communication link utilizes an interactive computer network. The method 300 also includes providing, a second communication link between a second clinic station 126 and the first medical assistant for medical information exchange. Yet, in one embodiment, the communication network 122 is established through an internet website accessible by the patient.

Various embodiments of the present disclosure provide a system for remote medical consultation. The system disclosed in the present disclosure provides remote access to a patient with a medical assistant by using the communication module and communication network. The system allows patients to remotely communicate with physicians, eliminating the need for physical visits in certain cases. Patients can connect with the physician from their own location, saving time and effort associated with travel and waiting times at the clinic. The system disclosed in the present disclosure utilizes the capabilities of the communication network and an interactive computer network to enable remote physician-patient consultations hence providing convenience of remote access to the medical assistant with the gathering of comprehensive patient information and facilitating efficient and effective healthcare services. Also, the system enables remote communication for more efficient interactions between physicians and patients and thereby reduces the number of visits required and allows for quicker access to medical advice and services.

Further, the system disclosed in the present disclosure enables the collection, storage, and retrieval of patient information in an electronic database. The patient's comprehensive information is collected by direct contact with the patient and stored and accessible to the medical assistant thereby enhancing the accuracy of assessments, diagnoses, and treatment plans. The system facilitates access to specialized medical expertise such as diet related medical assistant recommended by the diet recommendation module. Such specialized medical expertise enables remote consultations with medical assistants located in different geographic regions thereby expanding the options available to patients, ensuring they receive appropriate care and advice.

While specific language has been used to describe the disclosure, any limitations arising on account of the same are not intended. As would be apparent to a person skilled in the art, various working modifications may be made to the method in order to implement the inventive concept as taught herein.

The figures and the foregoing description give examples of embodiments. Those skilled in the art will appreciate that one or more of the described elements may well be combined into a single functional element.

Alternatively, certain elements may be split into multiple functional elements. Elements from one embodiment may be added to another embodiment. For example, the order of processes described herein may be changed and are not limited to the manner described herein. Moreover, the actions of any flow diagram need not be implemented in the order shown; nor do all of the acts need to be necessarily performed. Also, those acts that are not dependent on other acts may be performed in parallel with the other acts. The scope of embodiments is by no means limited by these specific examples.

We claim:

1. A system for remote medical consultation comprising:
a processing subsystem hosted on a server, and configured to execute on a network to control bidirectional communications among a plurality of modules comprising:
a communication module configured to remotely communicate a patient with a plurality of medical assistants;
a medical assistant recommendation module operatively connected to the communication module and configured to:
recommend a medical assistant from the plurality of medical assistants to the patient by generating a list of medical assistants consulted by the patient and a list of the medical assistants without consultation by the patient, wherein the recommendation is based on the historic data of medical assistant;
an examination module operatively coupled with the medical assistant recommendation module and configured to allow the medical assistant to remotely examine a plurality of health parameters of the patient, wherein the medical assistant is selected by the patient as recommended by a medical assistant recommendation module;
a diet recommendation module operatively connected with the examination module, wherein the diet recommendation module is configured to recommend a customized diet plan based on the plurality of health parameters of the patient; and
a template generation module operatively connected with the examination module, wherein the template generation module is configured to:
generate a template to allow the patient to fill a diagnostic data in a plurality of data fields and access a plurality of open platforms for generating the template; and
recommend the template to the patient based on an input from the medical assistant in a predefined data field, wherein the recommended template comprises of one or more customizable pre-filled data fields.

2. The system as claimed in claim 1, comprises a communication network comprising a first communication link between a first clinic station and the patient, wherein the first communication link utilizes an interactive computer network.

3. The system as claimed in claim 2, wherein the first clinic station comprises the plurality of medical assistants, wherein the patient requests remotely to the first clinic station for consultation with the medical assistant.

4. The system as claimed in claim 2, wherein the communication network comprises a second communication link between a second clinic station and the first medical assistant for medical information exchange with the first clinic station, wherein the second clinic station comprises a plurality of medical personnels.

5. The system as claimed in claim 2, wherein the first clinic station is located remotely from a patient station to communicate information relating to the patient.

6. The system as claimed in claim 3, wherein the second clinic station conducts an examination of the patient and collects a second set of information of the patient.

7. The system as claimed in claim 2, wherein the communication network is established through an internet website accessible by the patient.

8. The system as claimed in claim 1, comprises a database configured to store the data related to recommendations, wherein the database is accessible by the medical assistant.

9. The system as claimed in claim 1, wherein the template generation module is configured to automatically generate the template in real-time and notify a user as a notification.

10. A method for operating a system for a remote medical consultation comprising:
communicating, by a communication module of the processing subsystem, remotely by a patient with a plurality of medical assistants;
recommending, by a medical assistant recommendation module of a processing subsystem, a medical assistant from the plurality of medical assistants to a patient by generating a list of medical assistants consulted by the patient and a list of the medical assistants without consultation by the patient, wherein the recommendation is based on the historic data of medical assistant;
remotely examining, by an examination module of the processing subsystem, a plurality of health parameters the patient, wherein the medical assistant is selected by the patient as recommended by a medical assistant recommendation module;
recommending, by a diet generation module of the processing subsystem, a customized diet plan based on the plurality of health parameters of the patient;
generating, by a template generation module of the processing subsystem, generate a template to allow the patient to fill a diagnostic data in a plurality of data fields and accessing a plurality of open platforms for generating the template; and
recommending, by the template generation module of the processing subsystem, the template based on an input from the medical assistant in a predefined data field, wherein the recommended template comprises of one or more customizable pre-filled data fields.
